(19) United States
(12) Patent Application Publication
(10) Pub. No.: US 2025/0000771 A1
(43) Pub. Date: Jan. 2, 2025

(54) HAIR TREATMENT COMPOSITION COMPRISING KETAL/ACETAL OF GLYCERIN

(71) Applicant: L'OREAL, Paris (FR)

(72) Inventors: Jun LIANG, Brooklyn, NY (US); Heather LEE, Kinnelon, NJ (US)

(21) Appl. No.: 18/343,506

(22) Filed: Jun. 28, 2023

(51) Int. Cl. A61K 8/49; A61K 8/06; A61K 8/34; A61K 8/42; A61Q 5/12

(52) U.S. Cl. CPC A61K 8/4973; A61K 8/062; A61K 8/342; A61K 8/42; A61Q 5/12

(57) ABSTRACT

The instant disclosure is drawn to a hair treatment composition; and to methods for conditioning, managing, and improving the look and feel of hair. The hair treatment composition includes: (a) one or more ketal/acetal of glycerin compounds of Formula (I); (b) of one or more fatty alcohols; (c) one or more cationic surfactants; and (d) water. The compositions are typically oil-in-water emulsions or dispersions and are particularly useful in methods for detangling, smoothing, and moisturizing hair.

### FIELD OF THE DISCLOSURE

The instant disclosure relates to hair treatment compositions containing one or more ketal/acetal of glycerin compounds; and to methods for treating hair, for example, conditioning, managing, and improving the look and feel of hair.

### BACKGROUND

Many individuals suffer from dry and damaged hair. Dryness and damage can occur due to several factors including weather exposure, mechanical treatments (e.g., brushing hair), excessive treatments using chemicals, dying hair, heat styling, etc. In combination, using cleansing products that can be excessively stripping of hair's natural oils, can also lead to split ends, dull hair, and exacerbate dry hair. To mitigate the damage, oil treatments, conditioner, hair masks, and chemical treatments are commonly used.

The popularity and usage of oils for dry hair treatments has increased due to their effectiveness and simplicity. Commonly used oils include olive oil, mineral oil, avocado oil, apricot kernel oil, rice bran oil, and coconut oil. However, one problem is that effects are not usually seen after more than several hours (e.g., 8 hours) of treatment and several treatments are usually required, making it time consuming and labor intensive.

Individuals desire a treatment for hair or damaged hair that is not time consuming and labor intensive. A variety of approaches have been developed to condition the hair. A common method of providing conditioning benefit is through the use of conditioning agents such as cationic surfactants and polymers, high melting point fatty compounds, low melting point oils, silicone compounds, and combinations thereof. Most of these conditioning agents are known to provide various conditioning benefits.

However, there is still a need for providing improved hair conditioning benefits, for example, improved softness, improved hair alignment, increased ease of detangling, and increased shine.

### SUMMARY OF THE DISCLOSURE

The instant disclosure relates to hair treatment compositions that condition and improve the look and feel of hair; and to methods for conditioning and styling the hair using the compositions. Hair treated with the hair treatment compositions exhibits a significant improvement in the ease of detangling, regardless of whether the hair treatment compositions is applied before or after cleansing the hair with a shampoo. In addition, the hair treatment compositions provide a surprising degree of smoothness, slip, and softness to the hair.

The compositions typically include:
(a) one or more ketal/acetal of glycerin compounds of Formula (I):

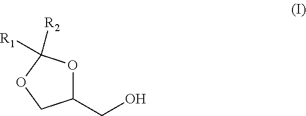

wherein R₁ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ hetero (C₃-C₆) cycloalkyl, aryl, heteroaryl, optionally substituted; or R₁ and R₂ may together form a C₃-C₆ cycloalkyl, C₃-C₆ heterocycloalkyl, aryl, or heteroaryl, optionally, substituted;
wherein heteroatoms are selected from N, O or S;
(b) one or more fatty alcohols having at least 8 carbon atoms;
(c) one or more cationic surfactants; and
(d) water.

The hair treatment compositions are typically oil-in-water emulsions or dispersions.

In various embodiments, the one or more ketal/acetal of glycerin compounds of Formula (I) are selected from compounds having at least one of R₁ and R₂ being a linear, branched, or cyclic C₁-C₆ alkyl. Preferably, R₁ and R₂ are independently a linear C₁-C₆ alkyl. An example of a particularly preferred ketal/acetal of glycerin compound is isopropylidene glycerol.

Nonlimiting examples of fatty alcohols having at least 8 carbon atoms include linear or branched fatty alcohols having from 10 to 30 carbon atoms, preferably from 12 to 28 carbon atoms. In various embodiments, the fatty alcohols are selected from capryl alcohol, pelargonic alcohol, decyl alcohol, undecyl alcohol, lauryl alcohol, tridecyl alcohol, myristyl alcohol, pentadecyl alcohol, cetyl alcohol, palmitoleyl alcohol, isostearyl alcohol, isocetyl alcohol, heptadecyl alcohol, stearyl alcohol, cetearyl alcohol, oleyl alcohol, nonadecyl alcohol, arachidyl alcohol, heneicosyl alcohol, behenyl alcohol, erucyl alcohol, lignoceryl alcohol, ceryl alcohol, 1-heptacosanol, montanyl alcohol, 1-nonacosanol, and myricyl alcohol.

Nonlimiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and combinations thereof.

In various embodiments, at least one of the one or more cationic surfactants is an amidoamine surfactant. Nonlimiting examples of amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palm itam idopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmito amidopropyl dimethylamine, stearamidoethyldiethylamine, or a mixture thereof.

In various embodiments, the hair treatment composition includes one or more non-silicone fatty compounds other than the one or more fatty alcohols of (b). Nonlimiting examples include oils, fatty acids, fatty esters, propylene glycol fatty acid esters, fatty carbonates, polyolefins (such as petrolatum), waxes, squalane, squalene, hydrogenated polyisobutene, hydrogenated polydecene, polybutene, mineral oil, pentahydrosqualene, plant and/or vegetable oil, hydrocarbon-based oils (such as isohexadecane), triglycerides, or a mixture thereof.

The hair treatment compositions optionally include one or more water-soluble solvents. Nonlimiting examples of water-soluble solvents include C₂-C₆ monoalcohols, polyols (polyhydric alcohols), glycerin, glycols, or a combination thereof. The polyols preferably have two or three hydroxyl groups. Nonlimiting examples of include ethylene glycol, propylene glycol, butylene glycol, hexylene glycol, pentylene glycol, 1,3-propanediol, diethylene glycol, dipropylene glycol, caprylyl glycol, glycerin, and a combination thereof.

In various embodiments, the hair treatment compositions include one or more thickening polymers. For example, the one or more thickening polymers may be nonionic thickening polymers, cationic thickening polymers, or a combination thereof. Nonlimiting examples of nonionic thickening polymers include polyacrylate polymers, polyacrylamide polymers, polysaccharides, gums (e.g., guar gums), and a combination thereof. The cationic thickening polymers include, but are not limited to, cationic polymers having quaternary amine group or a quaternary ammonium group.

The compositions can be applied immediately after shampooing the hair, for example, as a conditioner. The compositions can also be applied on hair immediately after shampooing and conditioning the hair, for example, as a mask rinse-off or leave-in treatment. The compositions can also be applied before shampooing the hair as pre-treatment composition. In various embodiments, after the composition is applied to the hair, the hair is rinsed with water, dried, and styled as desired. In other embodiments, the hair treatment composition is applied as a leave-on product. As a leave-on product, the hair treatment composition is applied to wet or damp hair and allowed to remain on the hair indefinitely, i.e., the hair treatment composition is not removed or rinsed from the hair prior to drying or styling the hair.

### DETAILED DESCRIPTION OF THE DISCLOSURE

The hair treatment compositions of the instant disclosure are particularly useful for conditioning hair, for example, by easing the detangling of the hair, smoothing and moisturizing the hair, and imparting slip and shine to the hair. Due to their unique ingredients and properties, the hair treatment compositions enhance the deposition of conditioning active agents, such as cationic surfactants and fatty compounds, onto or into the hair. The moisturizing and conditioning properties are long-lasting, resulting in hair having a light, healthy, and “managed” appearance. The compositions typically include:
(a) about 1 to about 25 wt. % of one or more ketal/acetal of glycerin compounds of Formula (I):

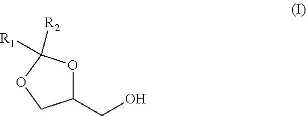

wherein R¹ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ hetero (C₃-C₆) cycloalkyl, aryl, heteroaryl, optionally substituted; or R₁ and R₂ may together form a C₃-C₆ cycloalkyl, C₃-C₆ heterocycloalkyl, aryl, or heteroaryl, optionally, substituted;
wherein heteroatoms are selected from N, O or S;
(b) about 1 to about 15 wt. % of one or more fatty alcohols;
(c) about 0.1 to about 10 wt. % of one or more cationic surfactants; and
(d) about 50 to about 92 wt. % of water;
   wherein all percentages by weight are based on a total weight of the composition.

In addition, in various embodiments, the hair treatment composition optionally includes one or more fatty compounds other than the fatty alcohol of (b), one or more thickening polymers, and/or one or more miscellaneous ingredients. The hair treatment compositions do not require silicones, although silicones can certainly be included in the compositions. Therefore, in various embodiments, the hair treatment compositions are free or essentially free from silicones.

The hair treatment compositions include water, fatty alcohols, and optionally one or more fatty compounds other than the fatty alcohols. Therefore, the compositions are typically oil-in-water emulsions or dispersions. The compositions typically have an opaque appearance and can have a creamy texture or a lotion-like consistency. The term “opaque” indicates that the composition is not transparent or translucent, i.e., has a transmittance of less than 50% at a wavelength of 600 nm, for example measured using a Lambda 40 UV-visible spectrometer.

### (a) Ketal/Acetal of Glycerin Compounds of Formula (I)

The hair treatment compositions include one or more ketal/acetal of glycerin compounds of Formula (I):

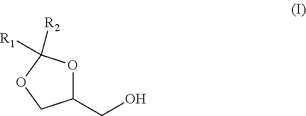

wherein
R₁ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ heterocycloalkyl, optionally substituted; or
R₁ and R₂ may together form a C₃-C₆ cycloalkyl, C₃-C₆ heterocycloalkyl, aryl, or heteroaryl, optionally substituted;
wherein heteroatoms are selected from N, O or S.

As used herein, the term “alkyl” is given its ordinary meaning in the art and includes saturated aliphatic groups, including straight-chain alkyl groups and branched-chain alkyl groups.

As used herein, the term “cycloalkyl” is given its ordinary meaning in the art and includes an alkyl as defined above but forming a ring having 3 to 6 carbon atoms.

As used herein, “alkenyl” is given its ordinary meaning in the art and includes straight chain and branched chain hydrocarbon groups containing at least one carbon-carbon double bond.

The term “heteroalkyl” is given its ordinary meaning in the art and includes an alkyl as described herein except that one or more atoms is a heteroatom (e.g., oxygen, nitrogen, sulfur, and the like).

As used herein, the term “heterocycloalkyl” is given its ordinary meaning in the art and includes a heteroalkyl as discussed above except in the form of a ring having 3 to 6 carbon atoms in which one or more atoms is a heteroatom (e.g., oxygen, nitrogen, sulfur, and the like).

As used herein, the term “heteroalkenyl” is given its ordinary meaning in the art and includes straight chain and branched chain hydrocarbon groups containing at least one carbon-carbon double bond and in which one or more atoms is a heteroatom (e.g., oxygen, nitrogen, sulfur, and the like).

As used herein, the term “aryl” is given its ordinary meaning in the art and includes aromatic carbocyclic groups having a single ring (e.g., phenyl) or multiple rings (e.g., biphenyl).

As used herein, the term “heteroaryl” is given its ordinary meaning in the art and includes aryl groups as described above except that one or more atoms is a heteroatom (e.g., oxygen, nitrogen, sulfur, and the like), optionally substituted.

The alkyl, alkenyl, heteroalkyl, heteraokenyl, aryl, heteroaryl, cycloalkyl, heterocycloalkyl groups discussed above may be optionally substituted, for example, optionally substituted with an alkyl, alkenyl, alkynyl, cycloalkyl, cycloalkenyl, cycloalkynyl, aryl, alkaryl, aralkyl, optionally interrupted or terminated by heteroatoms, carbonyl groups, cyano, NO₂, alkoxy, aryloxy, hydroxy, amino, thioalkyl, thioaryl, sulfur-containing groups, halides, substituted derivatives thereof, and the like.

In a preferred embodiment at least one of R₁ and R₂ is a linear, branched, or cyclic C₁-C₆ alkyl, preferably a linear or branched C₁-C₆ alkyl. In a further preferred embodiment, both R₁ and R₂ are both a linear, branched, or cyclic C₁-C₆ alkyl, preferably both a linear or branched C₁-C₆ alkyl.

Nonlimiting examples of ketal/acetal of glycerin compounds of Formula (I) include isopropylidene glycerol (2,2-dimethyl-1,3-dioxolane-4-methanol), (2,2-diethyl-1,3-dioxolan-4-yl) methanol, (2,2-dipropyl-1,3-dioxolan-4-yl) methanol, ((2-methyl-1,3-dioxolan-4-yl) methanol), (2-ethyl-1,3-dioxolan-4-yl) methanol, (2-propyl-1,3-dioxolan-4-yl) methanol, glycerol formal (4-hydroxymethyl-1,3-dioxolane), 2-isobutyl-2-methyl-1,3-dioxolane-4-methanol, 2-phenyl-1,3-dioxolane-4-methanol, and a combination thereof. Most preferably, the one or more ketal/acetal of glycerin is isopropylidene glycerol (2,2-dimethyl-1,3-dioxolane-4-methanol).

As used herein, the term “isopropylidene glycerol” is (2,2-dimethyl-1,3-dioxolan-4-yl) methanol, also known as “Solketal,” has the following formula:

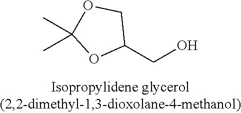

The total amount of the one or more ketal/acetal of glycerin compounds of Formula (I) will vary. Nonetheless, in various embodiments, the hair treatment composition includes about 1 to about 25 wt. % of the ketal/acetal of glycerin compounds of Formula (I), based on a total weight of the hair treatment composition. In further embodiments, the hair treatment composition includes about 1 to about 20 wt. %, about 1 to about 15 wt. %, about 1 to about 12 wt. %, about 1 to about 10 wt. %, about 1 to about 8 wt. %, about 2 to about 20 wt. %, about 2 to about 15 wt. %, about 2 to about 12 wt. %, about 2 to about 10 wt. %, about 2 to about 8 wt. %, about 3 to about 25 wt. %, about 3 to about 20 wt. %, about 3 to about 15 wt. %, about 3 to about 12 wt. %, about 2 to about 10 wt. %, about 3 to about 8 wt. %, about 4 to about 25 wt. %, about 4 to about 20 wt. %, about 4 to about 15 wt. %, about 4 to about 12 wt. %, about 4 to about 10 wt. %, about 4 to about 8 wt. %, or about 4 to about 6 wt. % of the one or more ketal/acetal of glycerin compounds of Formula (I), based on a total weight of the hair treatment composition.

### (b) Fatty Alcohols Having at Least 8 Carbon Atoms

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In various embodiments, the compositions include at least one solid fatty alcohol. Solid fatty alcohols are fatty alcohols that are solid at ambient temperature and at atmospheric pressure (25° C., 780 mmHg), and are insoluble in water, i.e., they have a water solubility of less than 1% by weight, preferably less than 0.5% by weight, at 25° C., 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include lauryl alcohol (1-dodecanol); myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), and combinations thereof. In a preferred embodiment, the compositions include at least one solid fatty alcohol selected from myristyl alcohol, cetyl alcohol, stearyl alcohol, behenyl alcohol and combinations thereof such as cetylstearyl or cetearyl alcohol.

In various embodiments, the compositions include at least one liquid fatty alcohol, in particular containing C10-C34 and preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched or straight alkyl group or an alkenyl group, R being optionally substituted by one or more hydroxy groups. In certain embodiments, the liquid fatty alcohols are selected from branched saturated alcohols. Preferably, R does not contain a hydroxyl group. Nonlimiting examples include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and combinations thereof. In other embodiments, the compositions are free or essentially free from liquid fatty alcohols, including the liquid fatty alcohols referenced above.

In a preferred embodiment, the one or more fatty alcohols are linear (straight chain) saturated fatty alcohols having from 10 to 30 carbon atoms, preferably from 12 to 28 carbon atoms, more preferably from 14 to 24 carbon atoms. Nonlimiting examples include decyl alcohol, undecyl alcohol, dodecyl alcohol, myristyl alcohol, cetyl alcohol, stearyl alcohol, cetearyl alcohol, behenyl alcohol, myricyl alcohol and a combination thereof.

The total amount of the one or more fatty alcohols in the hair treatment composition will vary. Nonetheless, in various embodiments, the hair treatment composition includes about 1 to about 15 wt. %, of the one or more fatty alcohols having at least 8 carbon atoms, based on a total weight of the hair treatment composition. In further embodiments, the hair treatment composition includes about 1 to about 12 wt. %, about 1 to about 10 wt. %, about 2 to about 15 wt. %, about 2 to about 12 wt. %, about 2 to about 10 wt. %, about 3 to about 15 wt. %, about 3 to about 12 wt. %, about 3 to about 10 wt. %, or about 3 to about 8 wt. %, based on a total weight of the hair treatment composition.

### (c) Cationic Surfactants

The term “cationic surfactant” means a surfactant that may be positively charged when it is contained in the compositions according to the disclosure. This surfactant may bear one or more positive permanent charges or may contain one or more functional groups that are cationizable in the composition according to the disclosure. Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and a combination thereof.

In various embodiments, the one or more cationic surfactants are preferably selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and a combination thereof.

In further embodiments, the one or more cationic surfactants are preferably selected from cetrimonium chloride, behentrimonium chloride, behentrimonium methosulfate, stearamidopropyl dimethylamine, and a combination thereof.

In a preferred embodiment, at least one of the one or more cationic surfactants include one or more amidoamine surfactants. Nonlimiting examples of amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, stearamidoethyldiethylamine, or a mixture thereof.

A more exhaustive but non-limiting list of cationic surfactants that may be included in the hair treatment compositions is provided later, under the heading “Additional Cationic Surfactants,” which is incorporated herein by reference.

The total amount of the one or more cationic surfactants in the composition will vary. Nonetheless, in various embodiments, the hair treatment composition includes about 0.1 to about 10 wt. % of the one or more cationic surfactants, based on a total weight of the hair treatment composition. In further embodiments, the hair treatment composition includes about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. % of the one or more cationic surfactants, based on a total weight of the hair treatment composition.

Weight Ratio of(a)/((b)+(c))

In various embodiments, the one or more ketal/acetal of glycerin compounds of Formula (I) of (a), the one or more fatty alcohols having at least 8 carbon atoms of (b), and the one or more cationic surfactants of (c) are in a weight ratio of (a) to ((b)+(c)) of about 0.5 or higher ((a)/((b)+(c))). In further embodiment, the weight ratio is about 0.5 to about 10, about 0.5 to about 8, about 0.5 to about 6, about 0.5 to about 4, about 0.5 to about 2, about 0.5 to about 1.5, about 0.5 to 1; about 0.6 to about 10, about 0.6 to about 8, about 0.6 to about 6, about 0.6 to about 4, about 0.6 to about 2, about 0.6 to about 1.5, or about 0.6 to about 1.

### (d) Water

The amount of water in the hair treatment compositions will vary depending on the amount of other ingredients in the hair treatment composition. Nonetheless, the hair treatment compositions typically include about 50 to about 92 wt. % water, based on a total weight of the hair treatment composition. In further embodiments, the hair treatment composition includes about 50 to about 90 wt. % water, about 55 to about 92 wt. %, about 55 to about 90 wt. %, about 55 to about 88 wt. % about 60 to about 92 wt. %, about 60 to about 90 wt. %, about 60 to about 88 wt. %, about 65 to about 92 wt. %, about 65 to about 90 wt. %, about 65 to about 88 wt. %, about 70 to about 92 wt. %, about 70 to about 90 wt. %, about 70 to about 88 wt. %, about 75 to about 92 wt. %, about 75 to about 90 wt. %, about 75 to about 88 wt. %, about 80 to about 92 wt. %, about 80 to about 90 wt. %, or about 80 to about 88 wt. %, based on a total weight of the hair treatment composition.

(e) Fatty Compounds Other than the One or More Fatty Alcohols of (b)

In various embodiments, the compositions of the instant disclosure include one or more fatty compounds other than the fatty alcohols of (b), preferably one or more non-silicone fatty compounds. The term “non-silicone fatty compound” means a fatty compound that does not containing any silicon atoms (Si). Nonlimiting examples of non-silicone fatty compounds include oils, mineral oil, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of fatty acids, fatty alcohol derivatives, and fatty acid derivatives are found in International Cosmetic Ingredient Dictionary, Sixteenth Edition, 2016, which is incorporated by reference herein in its entirety.

In various embodiments, the one or more fatty compounds other than the fatty alcohols of (b) comprise or consist of one or more liquid fatty compounds, also referred to “oils.” “Oil” is used herein to refer to an organic compound other than the fatty alcohols of (b) that is insoluble in water at normal temperature (25° C.) and at atmospheric pressure (760 mmHg), i.e. it has a water solubility of less than 5% by weight, or less than 1% by weight, or less than 0.1% by weight. Oils have in their structure a chain of at least two siloxane groups or at least one hydrocarbon chain having at least 6 carbon atoms. Furthermore, oils are generally soluble in organic solvents in the same conditions of temperature and pressure, for example in chloroform, ethanol, benzene or decamethylcyclopentasiloxane. Furthermore, oils are liquid at ordinary temperature (25° C.) and at atmospheric pressure (760 mmHg).

Fatty acids useful herein include those having from about 10 to about 30 carbon atoms, from about 12 to about 22 carbon atoms, and from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of polyglycerol esters of fatty acids include those of the following formula (II):

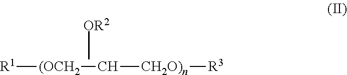

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. For example, nonionic polyglycerol esters of fatty acids include polyglyceryl-5 laurate.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and mixtures thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, 50° C. or higher. The high melting point fatty compound may be selected from the group consisting of fatty acids, fatty alcohol derivatives, fatty acid derivatives, and mixtures thereof. Nonlimiting examples of the high melting point compounds are found in the International Cosmetic Ingredient Dictionary, Sixteenth Edition, 2016, which is incorporated by reference herein in its entirety.

In some instances, fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

Mention may be made, among the waxes capable of being used as non-silicone fatty compounds, of animal waxes, such as beeswax, vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

i. Fatty Esters

Non-limiting examples of fatty esters include fatty esters from a C₆-C₃₂ fatty acid and/or a C₆-C₃₂ fatty alcohol. These esters may be esters of saturated or unsaturated, linear or branched C₁-C₂₆ aliphatic mono or polyacids and of saturated or unsaturated, linear or branched C₁-C₂₆ aliphatic mono or polyalcohols, the total number of carbon atoms in the esters being greater than or equal to 10. In some cases, for the esters of monoalcohols, at least one of the alcohol or the acid from which the esters result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate, glyceryl oleate, and isostearyl neopentanoate.

In various embodiments, the compositions of the instant disclosure may include cetyl esters. Cetyl esters are a mixture of the following esters of saturated fatty acids and fatty alcohols: cetyl palmitate, cetyl stearate, myristyl myristate, myristyl stearate, cetyl myristate, and stearyl stearate.

Mention is made of esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate, diisopropyl sebacate, diisopropyl adipate, di-n-propyl adipate, triisopropyl citrate, glyceryl trilactate, glyceryl trioctanoate, neopentyl glycol diheptanoate, and diethylene glycol diisononanoate.

Nonlimiting examples of liquid esters (ester oils) or liquid fatty esters that may be mentioned include, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, camellina oil, tamanu oil, babassu oil and pracaxi oil, jojoba oil, and shea butter oil, and caprylic/capric triglyceride.

Nonlimiting examples of solid fatty esters include solid esters obtained from C₉-C₂₆ fatty acids and from C₉-C₂₆ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

ii. Propylene Glycol Fatty Acid Esters

Nonlimiting examples of propylene glycol fatty acid esters include propylene glycol esters of medium chain fatty acids (fatty acids having from 6 to 12 carbon atoms), such as propylene glycol dicaprylate/dicaprate, propylene glycol dipelargonate, and propylene glycol dilaurate. A preferred propylene glycol fatty acid ester is propylene glycol dicaprylate/dicaprate. The term “propylene glycol dicaprylate/dicaprate” is understood by those in the art to refer to a combination containing propylene glycol dicaprylate, propylene glycol dicaprylate-caprate, and propylene glycol dicaprate, which may vary in the ratio of these components. An example of a commercially available form of propylene glycol dicaprylate/dicaprate is CAPTEX® 200, available from the Abitec Corp. (Columbus, OH, USA).

iii. Fatty Carbonate Esters

Nonlimiting examples of fatty carbonate esters include dialkyl carbonates of formula: R₁O(C═O) R₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C₁₄-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, and a combination thereof.

iv. Oils

Nonlimiting examples of oils include natural oils, such as coconut oil; hydrocarbons, such as mineral oil and hydrogenated polyisobutene; esters, such as C₁₂-C₁₅ alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid, or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols.

The total amount of the one or more non-silicone fatty compounds other than the fatty alcohols of (b), if present, will vary. Nonetheless, in various embodiments, the hair treatment composition includes about 0.1 to about 10 wt. % of one or more fatty compounds other than the fatty alcohols of (b), based on a total weight of the hair treatment composition. In further embodiments, the hair treatment composition includes about 0.1 to about 8 wt. %, about 0.1 to about 6 wt. %, about 0.1 to about 5 wt. %, about 0.1 to about 3 wt. %, about 0.1 to about 2 wt. %, about 0.1 to about 1 wt. %, about 0.5 to about 10 wt. %, about 0.5 to about 8 wt. %, about 0.5 to about 6 wt. %, about 0.5 to about 5 wt. %, about 0.5 to about 3 wt. %, about 0.5 to about 2 wt. %, about 0.5 to about 1 wt. % of one or more fatty compounds other than the fatty alcohols of (b), based on a total weight of the hair treatment composition.

### (f) Water-Soluble Solvent

In various embodiments, the hair treatment composition includes one or more water-soluble solvents. The term “water-soluble solvent” (or “water-soluble organic solvent”) is interchangeable with the term “water-miscible solvent.” Non-limiting examples of water-soluble organic solvents include glycerin, alcohols (for example, C₁₋₆ mono-alcohols), polyols, glycols, and a mixture thereof. In certain embodiments, the one or more water-soluble organic solvents are chosen from alcohols such as ethyl alcohol, isopropyl alcohol, propyl alcohol, and phenylethyl alcohol, or glycols or glycol ethers such as monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

In various embodiments, the hair treatment composition includes one or more water-soluble solvents chosen from short chain polyols (e.g., up to 6 carbon atoms) glycols, C₂₋₄ mono-alcohols, glycerin, and a mixture thereof; preferably the water-soluble organic solvent is chosen from glycerin, ethanol, isopropyl alcohol, dipropylene glycol, propylene glycol, butylene glycol, hexylene glycol, glycerin, or a mixture thereof.

The total amount of the one or more water-soluble solvents, if present, will vary. Nonetheless, in various embodiments, the hair treatment composition includes about 0.1 to about 25 wt. % of one or more water-soluble solvents, based on a total weight of the hair treatment composition. In further embodiments, the hair treatment composition includes about 0.1 to about 20 wt. %, about 0.1 to about 15 wt. %, about 0.1 to about 5 wt. %, about 0.1 to about 3 wt. %, about 1 to about 25 wt. %, about 1 to about 20 wt. %, about 1 to about 15 wt. %, about 1 to about 10 wt. %, or about 1 to about 5 wt. % of one or more water-soluble solvents, based on a total weight of the hair treatment composition.

### (g) Thickening Polymers

In certain embodiments, the hair treatment composition of the instant disclosure includes one or more thickening polymers. However, in other embodiments, the hair treatment compositions do not include thickening polymers, i.e., the composition is free or essentially free from thickening polymers. When included, in various embodiments, the one or more thickening polymers may be selected from nonionic thickening polymers, cationic thickening polymers, and a combination thereof.

The total amount of the one or more thickening polymers, if present, will vary.

Nonetheless, in various embodiments, the total amount of the one or more thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

i. Nonionic Thickening Polymers

In various embodiments, the compositions of the present disclosure include one or more nonionic thickening polymers. Nonlimiting examples of nonionic thickening polymers include methyl hydroxypropyl cellulose, gellan gum (such as “Kelcogel” from CP Kelco), polysaccharide, gum, hydroxyl propyl cellulose (such as “Methocel” from Dow/Amerchol), hydroxyl propyl methyl cellulose (such as “Klucel” from Hercules), hydroxyl ethyl cellulose, polyalkylene glycols, and combinations thereof. Particularly useful nonionic polymers include polysaccharide gum, hydroxyl propyl cellulose, hydroxyl propyl methyl cellulose, or combinations thereof.

The one or more nonionic thickening polymers may include polysaccharides, especially polysaccharides selected from modified or unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (such as methylcelluloses, hydroxyalkylcelluloses, and ethylhydroxyethylcelluloses), xylans including glucuronoxylans and arabinoxylans, glucans including xyloglucans, arabans, galactans including arabinogalactans, chitin, agars, locust bean gums, mannans including glucomannans and galactomannans such as guar gums and nonionic derivatives thereof (hydroxypropyl guar), and combinations thereof.

In a preferred embodiment, the one or more nonionic thickening polymers are selected from polyacrylate polymers, polyacrylamide polymers, polysaccharides, gums, and a combination thereof, more preferably selected from polysaccharides, gums, and combinations thereof, and even more preferably selected from guar gums, modified guar gums such as hydroxypropyl guar, and celluloses, for example, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and combinations thereof. In some instances, the cellulose is selected from water soluble cellulose derivatives (for example, methyl cellulose, methylhydroxypropyl cellulose, cetyl hydroxyethylcellulose, hydroxyethyl cellulose, hydroxypropyl cellulose, cellulose sulfate sodium salt), and combinations thereof. Furthermore, in some instance, the cellulose is preferably cetyl hydroxyethylcellulose.

The total amount of the one or more nonionic thickening polymers, if present, will vary. Nonetheless, in various embodiments, the total amount of the one or more nonionic thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

ii. Cationic Thickening Polymers

In various embodiments, suitable cationic thickening polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species. Anionic counterions can be used in association with the cationic thickening polymers. Non limiting examples of such counterions include halides (e.g., chloride, fluoride, bromide, iodide), sulfate, and methylsulfate. Non limiting examples of cationic thickening polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference in its entirety. More specifically, in various embodiments, the one or more cationic thickening polymer be selected from copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, or vinyl pyrrolidone.

Nonlimiting examples of cationic protonated amino and quaternary ammonium monomers, for inclusion in the cationic thickening polymers, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of useful cationic thickening polymers include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof.

In addition to the above, in certain embodiments, the one or more cationic thickening polymers may be selected from cationic polysaccharide polymers, such as cationic cellulose derivatives and cationic starch derivatives. Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their “Polymer LR,” “Polymer JR,” and “Polymer KG” series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename “Polymer LM-200.” Other suitable cationic thickening polymers include cationic guar gum derivatives, such as guar hydroxypropyltrimonium chloride, specific examples of which include the “Jaguar” series commercially available from Rhone-Poulenc Incorporated and the “N-Hance” series commercially available from Aqualon Division of Hercules, Inc. Other suitable cationic polymers include quaternary nitrogen-containing cellulose ethers.

In a preferred embodiment, the one or more cationic thickening polymers are selected from cellulose based cationic polymers, in particular, selected from polyquaternium-10, polyquaternium-24, polyquaternium-27, polyquaternium-67, polyquaternium-72, and a combination thereof.

The total amount of the one or more cationic thickening polymers, if present, will vary. Nonetheless, in various embodiments, the total amount of the one or more cationic thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

### (h) Miscellaneous Ingredients

The hair treatment compositions may optionally include one more miscellaneous ingredients. Miscellaneous ingredients are ingredients that are compatible with the hair treatment compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates and/or isolates, fillers (e.g., organic and/or inorganic fillers such as talc, calcium carbonate, silica, etc.) composition colorants, etc.

In the context of the instant disclosure, a “composition colorant” is a compound that colors the composition but does not have an appreciable coloring effect on hair. In other words, the composition colorant is included to provide a color to the composition for aesthetic appeal but is not intended to impart coloring properties to hair. As an example, hair styling gels, can be found in a variety of different colors (e.g., light blue, light pink, etc.) yet application of the styling gel to hair does not visibly change the color of the hair.

The total amount of the one or more miscellaneous ingredients, if present, will vary. Nonetheless, in various embodiments, the compositions include from about 0.001 to about 10 wt. % of one or more miscellaneous ingredients, based on the total weight of the composition. In further embodiments, the compositions include from about 0.001 to about 5 wt. %, about 0.001 to about 3 wt. %, about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. % of one or more miscellaneous ingredients, based on the total weight of the composition.

### FURTHER EMBODIMENTS

In various embodiments, the compositions of the instant disclosure may optionally include one or more of polyethylene glycols (PEG), anionic surfactants, anionic polymers, silicones including amine functionalized silicones, and combinations thereof. In other embodiments, the compositions of the instant disclosure are free or essentially free from one or more of polyethylene glycols (PEG), anionic surfactants, anionic polymers, silicones including amine functionalized silicones, and combinations thereof.

In various embodiments, the hair treatment composition includes one or more anionic surfactants. In other embodiments, the compositions do not include anionic surfactants, i.e., the compositions are free or essentially free from anionic surfactants. Anionic surfactants are known in the art. Nonlimiting examples of anionic surfactants include sulfate surfactants, isethionate surfactants, sarcosinate surfactants, sulfinate surfactants, taurate surfactants, etc. If present, the total amount of the one or more anionic surfactants may be from about 0.01 to about 5 wt. %, about 0.01 to about 3 wt. %, about 0.01 to about 1 wt. %, about 0.1 to about 4 wt. %, about 0.1 to about 2 wt. %, about 0.1 to about 1 wt. %, including ranges and sub-ranges therebetween, based on the total weight of the composition. In other embodiments, the compositions of the instant disclosure are free or essentially free from anionic surfactants.

In various embodiments, the compositions of the instant disclosure include one or more anionic polymers. In the context of the present disclosure, the term “anionic polymers” is understood as meaning those polymers which carry in a protic solvent under standard conditions at least one structural unit having permanently anionic groups, the anionic groups having to be compensated by counterions while maintaining electroneutrality. As contemplated herein, anionic groups are, for example, carboxylate, sulfate, or sulfonate groups. Nonlimiting examples of anionic polymers include anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and combinations thereof.

In a preferred embodiment, the compositions of the instant disclosure are free or essentially free from anionic polymers. For example, the compositions may be free or essentially free from any one or more of the anionic polymers referenced above. In addition, the compositions of the instant disclosure may be free or essentially free from one or more, or all of the anionic polymers selected from anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and a combination thereof.

In various embodiments, the compositions of the instant disclosure include one or more silicones. In other embodiments, the compositions are free or essentially free from silicones. Nonlimiting examples of silicones include dimethicone, dimethiconol, cyclomethicone, polysilicone-11, phenyl trimethicone, trimethylsilylamodimethicone, and stearoxytrimethylsilane. In a preferred embodiment, the one or more silicones are non-volatile silicon oils. Useful silicone oils include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicones, such as those with a viscosity 8 centistokes (8×106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or combinations thereof. In various embodiments, the hair treatment compositions include one or more silicone oils chosen from dimethicone, dimethiconol, cyclomethicone, polysilicone-11, phenyl trimethicone, and amodimethicone, and a combination thereof.

In various embodiments, the compositions include one or more amino functionalized silicones. In other embodiments, the compositions do not include one or more amino functionalized silicones, i.e., the compositions are free or essentially free from amino functionalized silicones. Nonlimiting examples of amino functionalized silicones include amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a combination thereof. Amodimethicone is a particularly useful amino functionalized silicone.

### PREFERRED EMBODIMENTS

In various embodiments, the hair treatment composition preferably comprises or consists of:
(a) about 1 to about 25 wt. %, preferably, about 2 to about 15 wt. %, more preferably about 3 to about 10 wt. % of one or more ketal/acetal of glycerin compounds of Formula (I):

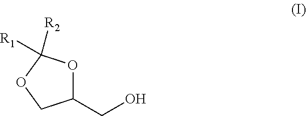

wherein R₁ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ hetero (C₃-C₆) cycloalkyl, aryl, heteroaryl, optionally substituted; or R₁ and R₂ may together form a C₃-C₆ cycloalkyl, C₃-C₆ heterocycloalkyl, aryl, or heteroaryl, optionally, substituted; wherein heteroatoms are selected from N, O or S; preferably, wherein at least one of R₁ and R₂ is a linear, branched, or cyclic C₁-C₆ alkyl; more preferably wherein R₁ and R₂ are independently a linear C₁-C₆ alkyl, for example, isopropylidene glycerol;
(b) about 1 to about 15 wt. %, preferably about 1 to about 10 wt. %, more preferably about 2 to about 8 wt. % of one or more fatty alcohols, wherein the one or more fatty alcohols are selected from linear or branched, saturated or unsaturated, C14-22 fatty alcohols, preferably wherein the one or more fatty alcohols are selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof;
(c) about 0.1 to about 10 wt. %, preferably about 0.1 to about 5 wt. %, more preferably about 0.5 to about 5 wt. % of one or more cationic surfactants, preferably wherein at least one of the one or more cationic surfactants is an amidoamine surfactant, preferably one or more amidoamine surfactants are selected from are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, stearamidoethyldiethylamine, or a mixture thereof;
   wherein (a), (b), and (c) are in a weight ratio of (a) to (b)+(c) of about of about 0.5 or higher, preferably about 0.5 to about 6, more preferably about 0.6 to about 3 ((a)/((b)+(c)));
(d) about 50 to about 92 wt. %, preferably about 65 to about 92 wt. %, more preferably about 70 to about 90 wt. % of water;
(h) optionally, about 0.01 to about 10 wt. %, preferably about 0.1 to about 8 wt. %, more preferably about 0.5 to about 5 wt. % of one or more miscellaneous ingredients
   wherein all percentages by weight are based on a total weight of the composition.

In further embodiments, the hair treatment composition preferably comprises or consists of:
(a) about 1 to about 25 wt. %, preferably, about 2 to about 15 wt. %, more preferably about 3 to about 10 wt. % of isopropylidene glycerol;
(b) about 1 to about 15 wt. %, preferably about 1 to about 10 wt. %, more preferably about 2 to about 8 wt. % of one or more fatty alcohols, wherein the one or more fatty alcohols are selected from linear or branched, saturated or unsaturated, C₁₄-22 fatty alcohols, preferably wherein the one or more fatty alcohols are selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof;
(c) about 0.1 to about 10 wt. %, preferably about 0.1 to about 5 wt. %, more preferably about 0.5 to about 5 wt. % of one or more cationic surfactants, preferably wherein at least one of the one or more cationic surfactants is an amidoamine surfactant, preferably one or more amidoamine surfactants are selected from are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, stearamidoethyldiethylamine, or a mixture thereof;
   wherein (a), (b), and (c) are in a weight ratio of (a) to (b)+(c) of about of about 0.5 or higher, preferably about 0.5 to about 6, more preferably about 0.6 to about 3 ((a)/((b)+(c)));
(d) about 50 to about 92 wt. %, preferably about 65 to about 92 wt. %, more preferably about 70 to about 90 wt. % of water;
(e) optionally, about 0.1 to about 10 wt. %, preferably about 0.1 to about 5 wt. %, more preferably about 0.1 to about 3 wt. % of one or more fatty compounds other than the one or more fatty alcohols of (b);
(f) optionally, about 0.1 to about 20 wt. %, preferably about 0.1 to about 10 wt. %, more preferably about 0.5 to about 5 wt. % of one or more water-soluble solvents, preferably wherein the one or more water-soluble solvents are selected from C₂-C₆ monoalcohols, polyols (polyhydric alcohols), glycerin, glycols, or a combination thereof;
(g) optionally, about 0.01 to about 5 wt. %, preferably about 0.1 to about 4, more preferably about 0.1 to about 3 wt. % of one or more thickening polymers;
(h) optionally, about 0.01 to about 10 wt. %, preferably about 0.1 to about 8 wt. %, more preferably about 0.5 to about 5 wt. % of one or more miscellaneous ingredients;
   wherein the composition has a pH of about 5 to about 8, preferably about 6 to about 8;
   the composition is an oil-in-water emulsion or dispersion; and
   all percentages by weight are based on a total weight of the composition.

The hair treatment composition set forth above may further include one or more film forming polymers. The one or more thickening polymers of (g), in addition to providing a thickening effect, may also provide a film forming effect. Therefore, in various embodiments, it may be preferable to include a thickening polymer that already has a film forming effect and, in some embodiments, it might be preferable to include a film forming polymer for purposes of forming a film on the hair, which may or may not have an appreciable influence on thickening.

### Viscosity

The compositions of the instant disclosure typically have a viscosity of about 10,000 mPa·s to about 150,000 mPa·s at 25° C. The viscosity measurements can be carried out, for example, using a Broooksfield viscometer, Model: DV−//+ Pro (Brookfield Engineering Laboratories, Inc.) at about 2 RPM, at ambient room temperature of about 20 to 25° C.; spindle sizes may be selected in accordance with the standard operating recommendations form the manufacturer, ranging from RV spindle No. 1 to No. 7. As noted above, one or more thickening polymers may optionally be included in the compositions, which will increase the viscosity of the compositions. In certain embodiments, the viscosity is about 10,000 mPa·s to about 100,000 mPa·s, about 20,000 mPa·s to about 100,000 mPa·s, or about 20,000 mPa·s to about 80,000 mPa·s.

pH

The pH of the hair treatment compositions according to the instant disclosure will vary. Nonetheless, in various embodiments, the hair treatment compositions have a pH of 5 to about 9. In further embodiments, the hair treatment composition has a pH of about 5 to about 8.5, about 5 to about 8, about 5.5 to about 9, about 5.5 to about 8, about 6 to about 9, or about 6 to about 8.

### Methods

The hair treatment compositions of the instant disclosure are useful for treating the hair, for example, for conditioning, managing, and improving the look and feel of hair. The hair treatment composition also also useful in methods for easing detangling of the hair. The compositions can be applied to the wet or damp hair and may be massaged into the hair, for example, with the hands, and/or spread throughout the hair with a comb or brush. The compositions can remain in the hair or can optionally be rinsed from the hair prior to drying and/or styling of the hair. Preferably, the hair treatment compositions are rinsed from the hair after being applied and massaged into the hair.

The hair treatment compositions, in certain embodiments, may be used as a leave-on product. The hair treatment compositions are applied to wet or damp hair and allowed to remain on the hair indefinitely, i.e., the hair composition is not removed or rinsed from the hair prior to drying or styling the hair.

The methods of treating hair according to the disclosure also include methods according to various routines. For instance, the compositions may be mixed with a shampoo (or conditioner) prior to application to the hair. Alternatively, the composition may be layered on top of (or lathered into) hair to which the shampoo (or conditioner) is already applied. Furthermore, the composition may be applied separate from the shampoo (or conditioner), i.e., applied to the hair after the shampoo (or conditioner) has been rinsed from the hair. In some instances, it is preferable to treat the hair with a composition of the instant disclosure prior to shampooing the hair, e.g., apply the composition to wet or damp hair prior to application of a shampoo to the hair. The hair may additionally (optionally) be treated with a conditioner after shampooing.

### Kits

The hair compositions of the instant disclosure may be incorporated into a kit. For example, the kits may include at least one hair treatment composition according to the instant disclosure and one or more additional compositions, for example, a shampoo, a conditioner, etc. The various compositions are separately contained in the kits. In some instances, the kits include one or more compositions according to the instant disclosure and a shampoo, which are separately contained. The kits may also include one or more compositions according to the instant disclosure, a shampoo, and a conditioner, all of which are separately contained. Instructions, mixing components, brushes, gloves, measuring tools, etc., may optionally be included in the kits.

The compositions may be packaged in a variety of different containers, such as, for example, a ready-to-use container. Non-limiting examples of useful packaging include tubes, jars, caps, unit dose packages, and bottles, including squeezable tubes and bottles. In some cases, the packaging is a tube, such as a tube with two compartments, or dual tubes, each forming a separate compartment. Each compartment may include a different composition. For example, one tube or compartment may include a composition according to the instant disclosure, and the other tube may include a composition to be used with the composition, for example, a shampoo, a conditioner, an all-in-one shampoo/conditioner (i.e., a conditioning shampoo; also referred to as a “co-wash”).

Methods of treating hair according to the disclosure will vary but typically include applying a hair treatment composition of the instant disclosure to wet or damp hair, allowing the composition to remain on the hair for a sufficient amount of time, and rinsing the compositions from the hair. The composition may be applied to the hair in a sequence with other compositions. For example, the compositions may be applied to the hair before shampooing the hair, after shampooing the hair, before conditioning the hair, and/or after conditioning the hair. The compositions, however, are not required to be used in a sequence.

The compositions of the instant disclosure may be allowed to remain on the hair for a minimum amount of time before being rinsed from the hair, but it is not necessary to allow the composition to remain on the hair. Conveniently, the compositions can be applied and allowed to remain on the hair for a period of time that is typical for regular shampooing and/or conditioning. For example, the composition (whether combined with another hair-treatment composition such as a shampoo or conditioner) may be applied to the hair and allowed to remain on the hair for a few seconds (1, 2, 3, or 5 seconds) up to about 1, about 2, about 5, about 10, about 15, about 20, about 25, or about 30 minutes.

The hair treatment composition may be applied to the hair immediately after or before the hair it treated with another composition (e.g., a shampoo and/or a conditioner). For example, the hair-treatment compositions may be applied to the hair within about 1, 2, 5, 10, or 20 minutes before or after a shampoo and/or a conditioner is applied to the hair.

The compositions of the instant disclosure are unique in their ability to provide hair with improved manageability and smoothness. Accordingly, the instant disclosure relates to methods for treating hair, for example, for improving the manageability of hair and for imparting smoothness. More specifically, the compositions may be used in methods for conditioning the hair, improving ease of combability and detangling, and/or providing smoothness.

### Additional Cationic Surfactants

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and combinations thereof.

In some cases, it is useful to use salts such as chloride salts of the quaternary ammonium compounds.

The fatty amines generally comprise at least one C₈-C₃₀ hydrocarbon-based chain.
A. Examples of quaternary ammonium salts that may especially be mentioned include those corresponding to the general formula (III) below:

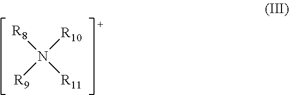

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur, and halogens. The aliphatic groups are chosen, for example, from C₁-C₃₀ alkyl, C₂-C₃₀ alkenyl, C₁-C₃₀ alkoxy, polyoxy (C₂-C₆)alkylene, C₁-C₃₀ alkylamide, (C₁₂-C₂₂)alkylamido (C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.
B. a quaternary ammonium salt of imidazoline, such as, for example, those of formula (IV) below:

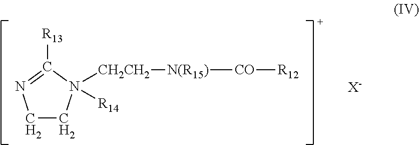

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a combination of alkenyl or alkyl groups containing from 12 to 21 carbon atoms, derived for example from tallow fatty acids, R₁₄ preferably denotes a methyl group, and R₁₅ preferably denotes a hydrogen atom. Such a product is sold, for example, under the name REWOQUAT W 75 by the company, Rewo;
C. a quaternary diammonium or triammonium salt, in particular of formula (V)

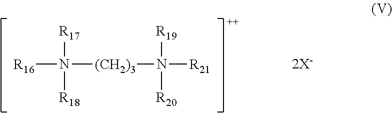

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ) (R₁₇ₐ) (R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, “Finquat CT-P”, sold by the company, Finetex (Quaternium 89), and “Finquat CT,” sold by the company, Finetex (Quaternium 75),
D. Cationic/Cationizable Surfactants, for Example of the General Structure

R4-A-R5-B
wherein R4 is a saturated or unsaturated, straight, or branched alkyl chain with 8 to 24 C atoms, R5 is a straight or branched alkyl chain with 1 to 4 C atoms, A is selected from:

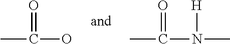

and B is selected from

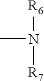

wherein R₆ and R₇ are the same or different is H or an alkyl chain with 1 to 4 C atoms, hydroxyl alkyl chain with 1 to 4 C atoms, and di hydroxyl alkyl chain with 2 to 4 C atoms,

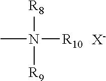

R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub.10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight, or branched alkyl chain with 10 to 24C atoms, more preferably 12 to 22 C atoms and R₅ is straight or branched alkyl group with 1 to 4 C atoms, and A, B, R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants or amphiphilic surfactants may be chosen from fatty alkylamines, preferably, fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and combinations thereof. In various embodiments, it is preferable that at least one of the one or more cationic surfactants is a fatty dialkylamine.

Fatty dialkylamines include fatty amidoamine compounds, their salts, and combinations thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine. In a preferred embodiment, the compositions of the instant disclosure include stearamidopropyl dimethylamine, and optionally one or more additional cationic surfactants.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and combinations thereof. In particular, lactic acid or tartaric acid or combinations thereof, are useful, especially in combination with fatty dimethylamines such as, for example, stearamidopropyl dimethylamine.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

| | | | Inventive | | | | | | | | | Comparative | | |
|---|---|---|---|---|---|---|---|---|---|---|---|---|---|---|
| | | | A | B | C | D | E | F | G | H | I | J | K | L |
| (a) | Formula (I) | Isopropylidene Glycerol | 5 | 5 | 5 | 5 | 5 | 5.5 | 5.5 | 5.5 | 5.5 | | 5 | 5 |
| (b) | Fatty Alcohol | Myristyl Alcohol | 2 | 2 | 2 | 2 | 2 | 2.2 | 1.7 | 1.7 | 1.7 | 2 | | 2 |
| | | Cetearyl Alcohol | 2 | 2 | 2 | 2 | 2 | 2.2 | 2.8 | 2.8 | 2.8 | 2 | | 2 |
| | | Oleyl Alcohol | | | | | | | 1.1 | 1.1 | 3.3 | | | |
| Total Fatty Alcohol | | | 4 | 4 | 4 | 4 | 4 | 4.4 | 5.5 | 5.5 | 7.7 | 4 | | 4 |
| | Cationic | Brassicamidopropyl | 1 | 1 | 1 | 1 | 1 | 1.1 | 1.1 | 1.1 | 1.1 | 1 | 1 | |
| | Surfactant | Dimethylamine | | | | | | | | | | | | |
| Total (b) + (c) | | | 5 | 5 | 5 | 5 | 5 | 5.6 | 6.6 | 6.6 | 8.8 | 5 | 1 | 4 |
| Ratio (a)/((b) + (c) | | | 0.8 | 0.8 | 0.8 | 0.8 | 0.8 | 0.8 | 0.8 | 0.8 | 0.9 | 0.8 | NA | 0.8 |
| (d) | Water | Water | QS | QS | QS | QS | QS | QS | QS | QS | QS | QS | QS | QS |
| (e) | Fatty | Coconut Oil | | 1 | | | | | 0.5 | 0.5 | 0.5 | | | |
| | Compound | Glyceryl Oleate | | | 1 | | | | | | | | | |
| | | Isopropyl Myristate | | | | 1 | | | | | | | | |
| | | Capric/Caprylic | | | | | 1 | | | | | | | |
| | | Triglyceride | | | | | | | | | | | | |
| (f) | Optional Water- | | | | | | | | | | | | | |
| | Soluble Solvent | | | | | | | | | | | | | |
| (g) | Optional | Cetyl Hydroxoxyethyl | | | | | | | | 0.1 | | | | |
| | Thickening | Cellulose | | | | | | | | | | | | |
| | Polymer | | | | | | | | | | | | | |
| (h) | Misc.¹ | pH adjusters, salts, | ≤5 | ≤5 | ≤5 | ≤5 | ≤5 | ≤5 | ≤5 | ≤5 | ≤5 | ≤5 | ≤5 | ≤5 |
| | | fragrances, preservatives, | | | | | | | | | | | | |
| | | antioxidants, chelating | | | | | | | | | | | | |
| | | agents, compositions | | | | | | | | | | | | |
| | | colorants, fillers, | | | | | | | | | | | | |
| | | humectants, emollients, | | | | | | | | | | | | |
| | | botanical extracts, etc. | | | | | | | | | | | | |

| | | |
|---|---|---|
| Viscosity² | 30,000-100,000 | <100 |

| | |
|---|---|
| pH | 6-7 |

¹For example, hydrated silica, silica dimethyl silylate, benzoic acid, lactic acid, tartaric acid, disodium phosphate, sodium phosphate, hydrogenated palm glycerides citrate, ascorbyl palmitate, tocopherol, lecithin, phenoxyethanol, caprylyl glycol and/or fragrances, etc.
²Measured at 25° C. measured with a Brookfield DV-LL + Pro Viscometer using RV spindles and rotational speed of 2 RPM.

### Example 2

Equal amount of Inventive Composition A and Comparative Composition J of Example 1 was applied to Curl Pattern 6 natural hair swatches and the swatches were allowed to air dry. After the hair swatches were dry, the swatches were visually accessed. The hair treated with Inventive Composition A displayed significantly less residue and flaking than the hair treated with Comparative Composition J.

EXAMPLE 3
Inventive Composition G from Example 1 was
tested on hair on the heads of three human volunteers
having medium-long, fine-average, very curly, natural-low
damage hair, and compared to a Commercial Benchmark Product.
Inventive Composition G is shown in the table below.

| | | | G |
|---|---|---|---|
| (a) | Formula (I) | Isopropylidene Glycerol | 5.5 |
| (b) | Fatty Alcohol | Myristyl Alcohol | 1.7 |
| | | Cetearyl Alcohol | 2.8 |
| | | Oleyl Alcohol | 1.1 |
| Total Fatty Alcohol | | | 5.5 |
| (c) | Cationic | Brassicamidopropyl | 1.1 |
| | | Dimethylamine | |
| | Surfactant | Behentrimonium Chloride and | |
| | | Cetrimonium Chloride | |
| Total (b) + (c) | | | 6.6 |
| Ratio (a)/((b) + (c)) | | | 0.8:1 |
| (d) | Water | Water | 85 |
| (e) | Fatty | Coconut Oil | 0.5 |
| | Compound | Vegetable Oil | 0.4 |
| | Silicon | Dimethicone and | |
| | | Amodimethicone | |
| (f) | Water-Soluble | Glycerin, Propylene Glycol, | |
| | Solvent | and Isopropyl Alcohol | |
| (h) | Misc.¹ | pH adjusters, salts, fragrances, | ≤5 |
| | | preservatives, antioxidants, | |
| | | chelating agents, compositions | |
| | | colorants, fillers, humectants, | |
| | | emollients, botanical extracts, etc. | |

| | |
|---|---|
| Viscosity¹ | 42,000 |
| pH² | 6.5 |

¹pH adjusters, salts, fragrances, preservatives, antioxidants, chelating agents, compositions colorants, fillers, humectants, emollients, botanical extracts, etc. For example, hydrated silica, silica dimethyl silylate, lactic acid, tartaric acid, disodium phosphate, sodium phosphate, hydrogenated palm glycerides citrate, ascorbyl palmitate, tocopherol, lecithin, phenoxyethanol, caprylyl glycol and/or fragrances, etc.
²Measured at 25° C. measured with a Brookfield DV-LL+ Pro Viscometer using RV Spindle and rotational speed of 2 RPM.

The Commercial Benchmark includes the following ingredients listing: water, cetearyl alcohol, dimethicone, amodimethicone, behentrimonium chloride, cetrimonium chloride, glycerin, propylene glycol, isopropyl alcohol, and miscellaneous ingredients.

Equal amounts of Inventive Compositions G and Commercial Benchmark Product were applied to opposite halves of a head of dry hair and the hair was independently evaluated for ease of pre-shampoo detangling and amount of slip by an expert stylist. Ease of detangling was assessed by detangling the hair with wide tooth comb. The term “slip” refers to the “slippery-ness” or “lubrication” of the hair, determined by running the hair through the fingers.

Hair treated with Inventive Compositions G was consistently rated higher for both pre-shampoo ease of detangling and slip than the Commercial Benchmark Product.

After pre-shampoo detangling, the hair was cleansed with a standard shampoo. The shampoo was rinsed from the hair and Inventive Compositions G and the Commercial Benchmark Product were applied to opposite halves of a head of damp hair as a leave-on treatment. Without drying, the hair was independently evaluated for ease of detangling and amount of slip by an expert stylist.

After the hair was evaluated for ease of detangling and slip, it was blow dried and evaluated for smoothness, moisturization, and flaking by an expert stylist. Hair treated with Inventive Compositions G was consistently rated better for smoothness (more smoothness), moisturization (more moisturization), and flaking (less flaking).

The above findings are significant considering Inventive Composition G does not include silicones (e.g., dimethicone and amodimethicone). Silicones are often used in conditioning compositions to ease detangling, provide slip, and improve smoothness and moisturization. The data show that the inventive compositions containing a ketal/acetal of glycerin (isopropylidene glycerol) as opposed to silicones provided better conditioning properties (ease of detangling, slip, smoothness, moisturization, and reduced flaking) than a commercial benchmark product formulated and marketed for its conditioning properties. Therefore, it was particularly unexpected to find that the inventive compositions provided better conditioning properties than the commercial benchmark product containing silicones.

The terms “comprising,” “having,” and “including” are used in their open, non-limiting sense.

The terms “a” and “the” are understood to encompass the plural as well as the singular.

The compositions and methods of the present disclosure can comprise, consist of, or consist essentially of the essential elements and limitations of the disclosure described herein, as well as any additional or optional ingredients, components, or limitations described herein or otherwise useful.

A “composition colorant” is a compound or ingredient that colors the composition but does not have an appreciable coloring effect on hair. In other words, the composition colorant is included to provide a color to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Thus, a composition colorant is different from a hair coloring agent. A composition colorant colors the compositions; a hair coloring agent may change the color of the compositions (and often does) but also changes the color of hair

The term “rinse-off” as used herein indicates that the composition is used in a context whereby the composition is ultimately rinsed or washed from the treated surface, (e.g., skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

A “lamellar phase” refers generally to packing of polar-headed long chain nonpolar-tail molecules in an environment of bulk polar liquid (i.e., water from the hair), as sheets of bilayers separated by bulk liquid.

The term “non-linear fatty acids” as used in the instant disclosure refers to unsaturated fatty acid and/or branched fatty acids.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

As used herein, the expression “at least one” is interchangeable with the expression “one or more” and thus includes individual components as well as mixtures/combinations.

The term “substantially free” or “essentially free” as used herein means that there is less than about 5% by weight of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair.

Throughout the disclosure, the term “a combination thereof” (or a mixture thereof) may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. In other words, even if the expression “a salt thereof” is not specifically or expressly stated with respect to ingredients that can form salts or are available as salts, it is understood that the salt form of the compound is included.

The expression “inclusive” for a range of concentrations means that the limits of the range are included in the defined interval.

“Volatile,” as used herein, means having a flash point of less than about 100° C.

“Non-volatile,” as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping component or ingredient does not represent more than one component. For example, if a polyacrylate falls within the description of a thickening polymer and within the description of film forming polymer, the specifically recited polyacrylate is understood as being only the thickening polymer or only the film forming polymer. A specific polyacrylate cannot simultaneously be construed as both a thickening polymer and a film forming polymer, for example, when the claims set forth both a thickening polymer and a film forming polymer (even though the polyacrylate may function as both a thickening polymer and a film forming polymer). In other words, a single compound or ingredient cannot simultaneously serve as two different components of a claimed composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 1 to about 25 wt. % of one or more ketal/acetal of glycerin compounds of Formula (I): 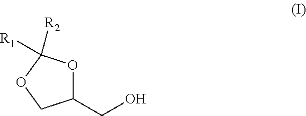
wherein R₁ and R₂ can be the same or different and are independently selected from hydrogen, C₁-C₆ alkyl, C₁-C₆ alkenyl, C₁-C₆ heteroalkyl, C₁-C₆ heteroalkenyl, aryl, heteroaryl, C₃-C₆ cycloalkyl, C₃-C₆ hetero (C₃-C₆) cycloalkyl, aryl, heteroaryl, optionally substituted; or R₁ and R₂ may together form a C₃-C₆ cycloalkyl, C₃-C₆ heterocycloalkyl, aryl, or heteroaryl, optionally substituted;
wherein heteroatoms are selected from N, O or S;
(b) about 1 to about 15 wt. % of one or more fatty alcohols;
(c) about 0.1 to about 10 wt. % of one or more cationic surfactants; and
(d) about 50 to about 92 wt. % of water;
wherein all percentages by weight are based on a total weight of the composition.

2. The composition of claim 1, wherein at least one of R₁ and R₂ is a linear, branched, or cyclic C₁-C₆ alkyl.

3. The composition of claim 1, wherein R₁ and R₂ are independently a linear C₁-C₆ alkyl.

4. The composition of claim 1, wherein the ketal/acetal of glycerin compound of Formula (I) is isopropylidene glycerol.

5. The composition of claim 1, wherein the one or more fatty alcohols are selected from linear or branched, saturated or unsaturated, C10-22 fatty alcohols.

6. The composition of claim 1, wherein the one or more fatty alcohols are selected from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, and a mixture thereof.

7. The composition of claim 1, wherein (a), (b), and (c) are in a weight ratio of (a) to (b)+(c) of about 1:2 to about 10:1 ((a):((b)+(c))).

8. The composition of claim 1, wherein at least one of the one or more cationic surfactants is one or more amidoamine surfactants.

9. The composition of claim 8, wherein the one or more amidoamine surfactants are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, stearamidoethyldiethylamine, or a mixture thereof.

10. The composition of claim 1 having a pH from about 5 to about 9.

11. The composition of claim 1, wherein the composition is an oil-in-water emulsion or dispersion.

12. The composition of claim 1, wherein the composition is opaque and creamy in texture.

13. The composition of claim 1, further comprising:
(e) one or more non-silicone fatty compounds other than the one or more fatty alcohols of (b).

14. The composition of claim 1, further comprising:
(f) one or more water-soluble solvents.

15. The composition of claim 14, wherein the one or more water-soluble solvents are selected from C₂-C₆ monoalcohols, polyhydric alcohols, glycols, or a combination thereof.

16. The composition of claim 1, wherein the composition is free or essentially free from silicones.

17. A hair treatment composition comprising:
(a) about 2 to about 20 wt. % of isopropylidene glycerol;
(b) about 1 to about 10 wt. % of one or more linear or branched, saturated or unsaturated, C14-22 fatty alcohols;
(c) about 0.5 to about 5 wt. % of one or more amidoamine surfactants;
wherein (a), (b), and (c) are in a weight ratio of (a) to (b)+(c) of about 0.5 or higher;
(d) about 70 to about 90 wt. % of water;
(e) optionally, about 0.01 to about 5 wt. % of one or more non-silicone fatty compounds other than the one or more fatty alcohols of (b);
(f) optionally, about 0.01 to about 5 wt. % of one or more water-soluble solvents;
(g) optionally, about 0.01 to about 5 wt. % of one or more thickening polymers; and
(h) optionally, about 0.01 to about 5 wt. % of one or more miscellaneous ingredients; wherein,
the composition has a pH from about 5 to about 8;
the composition is an oil-in-water emulsion or dispersion; and
all percentages by weight are based on a total weight of the composition.

18. A method for treating hair comprising applying the hair treatment composition of claim 1 to the hair.

19. The method of claim 18, wherein application of the hair treatment compositions improves smoothness, moisturization, and/or slip of the hair.

20. The method of claim 18, wherein the hair treatment composition is applied to the hair and the hair is dried or styled without rinsing the composition from the hair prior to drying or styling.
